(19) United States
(12) Patent Application Publication
(10) Pub. No.: US 2007/0154424 A1
(43) Pub. Date: Jul. 5, 2007

(54) Cosmetic

(75) Inventors: Yoshikazu Konno, Funabashi-shi (JP); Masahiro Moriyama, Funabashi-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kose Corporation, Tokyo (JP)

(21) Appl. No.: 10/586,630

(22) PCT Filed: Jan. 19, 2005
(86) PCT No.: PCT/JP05/00583

§ 371 Date: Jul. 19, 2006

(51) Int. Cl. A61K 8/55; A61K 8/26

(52) U.S. Cl. 424/62

(57) ABSTRACT

A cosmetic containing L-ascorbic acid 2-phosphate sodium salt that excels in long-term stability. In particular, there is provided a cosmetic characterized in that L-ascorbic acid 2-phosphate sodium salt (A) and at least one compound (B) selected from the group consisting of arginine, urea and triethanolamine are contained in a mixing mass ratio of (A):(B) ranging from 1:0.001 to 1:0.5.

### TECHNICAL FIELD

The present invention relates to a cosmetic comprising L-ascorbic acid 2-phosphate sodium salt, particularly, to a cosmetic that, as a result of comprising specific components, prevents yellowing and a strange odor resulting from decomposition of L-ascorbic acid 2-phosphate sodium salt and possesses excellent storage stability.

### BACKGROUND ART

Conventionally, with an objective of improving or preventing darkening of the skin resulting from sunburns and the like and spots, freckles, and the like resulting from pigmentation, skin lightening cosmetics and anti-aging cosmetics such as a skin lotion, milky lotion, cream, beauty solution, pack, cleansing preparation, dispersion, ointment, solution, aerosol, adhesive skin patch, and the like are used, of which a majority comprise L-ascorbic acid as an active component.

However, since L-ascorbic acid is comparatively easily oxidized and hydrolyzed, it easily decomposes when used in a cosmetic as is and degrades the quality of the cosmetic by decreasing the skin lightening effect of improving and preventing spots and freckles, causes yellowing of the cosmetic, and produces a strange odor. Therefore, in order to use L-ascorbic acid in cosmetics, various derivatives obtained by chemically modifying L-ascorbic acid to improve its stability are used.

L-ascorbic acid 2-phosphate sodium salt is one such derivative developed to improve the storage stability of L-ascorbic acid. Although L-ascorbic acid 2-phosphate sodium salt has significantly higher storage stability than L-ascorbic acid, it does not possess sufficient storage stability as a cosmetic because yellowing and a strange odor resulting from decomposition of the L-ascorbic acid 2-phosphate sodium salt tends to occur in the cosmetic after a long period of time, particularly when stored at a temperature of 30° C. or more. There is also a concern of decreased usability in regard to the skin lightening effect of the cosmetic.

With an objective of further improving storage stability of cosmetics comprising L-ascorbic acid 2-phosphate sodium salt, a method of using L-ascorbic acid 2-phosphate sodium salt in combination with a salt having a valence of two or more (Japanese Patent Application Laid-open No. 1997-118613), a method of using L-ascorbic acid 2-phosphate sodium salt in combination with a compound having a thiol group or a disulfide bond, sulfurous acid, and an amino acid having a hydroxyl group (PCT Publication No. WO 01/043702), and the like have been proposed.

However, when L-ascorbic acid 2-phosphate sodium salt is combined with a salt having a valence of two or more, yellowing and a strange odor resulting from decomposition of the L-ascorbic acid 2-phosphate sodium salt when stored for a long period of time, particularly at a high temperature of 30° C. or more, is not sufficiently controlled. Although yellowing resulting from decomposition of L-ascorbic acid 2-phosphate sodium salt can be inhibited by combining L-ascorbic acid 2-phosphate sodium salt with a compound having a thiol group or a disulfide bond, sulfurous acid, and an amino acid having a hydroxyl group, the strange odor is not sufficiently inhibited. Furthermore, there has been a problem of a strange odor when a compound comprising a thiol group or disulfide bond is used as a storage stabilizer in a cosmetic stored at a high temperature of 30° C. or more, which is believed to originate from the storage stabilizer itself.

### DISCLOSURE OF THE INVENTION

### Problems to be Solved by the Invention

Development of a method for preventing decomposition of L-ascorbic acid 2-phosphate sodium salt in a cosmetic stored for a long period of time under high temperature conditions has been desired.

### MEANS FOR SOLVING THE PROBLEMS

As a result of extensive studies, the present inventors discovered that yellowing and a strange odor resulting from decomposition of the L-ascorbic acid 2-phosphate sodium salt can be controlled and long term stability of the cosmetic can be maintained by adding a specific amount of a specific nitrogen-containing compound to a cosmetic comprising L-ascorbic acid 2-phosphate sodium salt, thereby completing the present invention.

Specifically, the present invention provides a cosmetic comprising the following components (A) and (B):

(A) L-ascorbic acid 2-phosphate sodium salt,

(B) one or more compounds selected from the group consisting of arginine, urea, and triethanolamine,

at a mass ratio of (A):(B) of 1:0.001 to 0.5.

The present invention also provides the above cosmetic further comprising the following component (C):

(C) zinc oxide or a salt which produces an alkali metal ion in water, at a mass ratio of (A):(C) of 1:0.001 to 1.

### EFFECT OF THE INVENTION

The cosmetic of the present invention exhibits excellent storage stability in spite of the inclusion of L-ascorbic acid 2-phosphate sodium salt, whereby yellowing and strange odor are suppressed even when stored for a long period of time at a high temperature. Therefore, the cosmetic of the present invention can be used in a wide variety of cosmetics such as a skin lightening cosmetic and anti-aging cosmetic.

### BEST MODE FOR CARRYING OUT THE INVENTION

The L-ascorbic acid 2-phosphate sodium salt used as the component (A) of the present invention is a substance known to be used in cosmetics for its skin lightening effect of improving and/or preventing skin darkening resulting from sun tanning, skin dullness, spots, and freckles or skin pigmentation such as senile pigment freckles and liver spots or its anti-aging effect of preventing decrease in skin elasticity resulting from sun tanning, sagging of the skin, and wrinkles. Although there are no limitations to the amount of the component (A) to be used in the cosmetic of the present invention, an amount of 0.001 to 20 mass % (hereinafter simply referred to as “%”) of the total amount of the components is preferable, with 0.1 to 5% being even more preferable.

The arginine, urea, and triethanolamine used as the component (B) of the present invention are incorporated to increase the storage stability of the component (A) and can be selected from components commonly used in cosmetics. The component (B) is incorporated in the cosmetic of the present invention in such an amount that the mass ratio of the component (A) to the component (B) is 1:0.001 to 0.5 and preferably 1:0.05 to 0.3. If the mass ratio of the component (B) to the component (A) is less than 0.001, a sufficient storage stability effect cannot be obtained, and if the mass ratio of the component (B) exceeds 0.5, the storage stability does not increase further.

The combination of arginine and urea as the component (B) is preferable due to the significant increase in storage stability of the L-ascorbic acid 2-phosphate sodium salt. Although there are no specific limitations to the mass ratio of arginine:urea, 1:10 to 10:1 is preferable due to the particular increase in storage stability.

Although yellowing and a strange odor can be effectively inhibited by only the addition of the component (B) in the present invention, zinc oxide or a salt which produces an alkali metal ion in water of the component (C) is preferably further added to obtain a more excellent effect.

Any zinc oxide commonly used in cosmetics can be used as the component (C) in combination with the component (B) to further increase the storage stability of the component (A). As a salt which produces an alkali metal ion in water of the component (C), sodium chloride, potassium chloride, potassium citrate, sodium citrate, sodium lactate, sodium succinate, disodium succinate, sodium malate, sodium aspartate, potassium aspartate, potassium sulfate, sodium sulfate, potassium carbonate, sodium carbonate, sodium hydrogencarbonate, sodium monohydrogenphosphate, sodium dihydrogenphosphate, potassium monohydrogenphosphate, potassium dihydrogenphosphate, and the like can be given, with potassium chloride, sodium chloride, and the like being preferable.

The component (C) is contained in the cosmetic of the present invention in an amount so that the mass ratio of the component (A) to the component (C) is 1:0.001 to 1 and preferably 1:0.01 to 0.5. If the mass ratio of the component (C) to the component (A) is less than 0.001, an increase in the storage stability effect would be difficult to obtain, and if the mass ratio of the component (C) exceeds 1, the storage stability does not increase further. Therefore, the mass ratio of component (A):component (B):component (C) is 1:0.001 to 0.5:0.001 to 1 and preferably 1:0.05 to 0.3:0.01 to 0.5.

When necessary, various components commonly used in cosmetics, quasi-drugs, medical supplies, and the like other than the above components may be suitably added to the cosmetic of the present invention. As examples of these optional components, alcohols, humectant, oily component, emulsifier, emulsification stabilizer, thickener, antiseptic agent, powder other than the zinc oxide of component (C), pigment, dye, UV absorber, pH adjuster, perfume, medicinal components other than the component (A), and the like can be given.

The cosmetic of the present invention can be prepared by a common method in accordance with the form of the cosmetic. As examples of the form of the cosmetic, milky lotion, lotion, cream, pack, stick, cleansing preparation, make-up, dispersion, ointment, solution, aerosol, adhesive skin patch, and the like can be given, with milky lotion and aqueous type cosmetics being preferable. The cosmetic of the present invention as described above is preferably used as a skin lightening cosmetic and anti-aging cosmetic.

The cosmetic of the present invention preferably has a pH in a range of 7 to 9. If the pH is lower than 7, yellowing and a strange odor occur easily and storage stability is inferior. On the other hand, if the pH is greater than 9, safety to the skin may decrease.

The cosmetic of the present invention is not limited to common skin cosmetics but can be used in all types of agents for use on the skin such as quasi-drugs, medical supplies, and the like.

### EXAMPLES

The present invention will be described in more detail by examples, which should not be construed as limiting the present invention.

### Example 1

Cream:

Creams with the composition shown in Table 1 were prepared by the following method. The creams obtained were evaluated by the following storage stability examination (1) and storage stability examination (2). The results are shown in Table 1.

(Method of Preparation)

A. Components (1) to (6) were mixed, heated, and the mixture was allowed to stand at 70° C.

B. Components (7) to (16) were heated and the mixture was allowed to stand at 70° C.

C. B was added to A and the mixture was emulsified and cooled to obtain a cream.

<Storage Stability Examination (1)>

Samples of each of the creams were stored for one month in a thermostatic chamber at 50° C. and the remaining amounts of L-ascorbic acid 2-phosphate sodium salt were measured using high-performance liquid chromatography. The results show the remaining amount as a percentage of the amount when the sample was prepared.

<Storage Stability Examination (2)>

Two samples of each cream were filled into identical glass containers. One of the glass containers was placed in a thermostatic chamber at 5° C. and the other was placed in a thermostatic chamber at 40° C. and stored for six months. Change in outer color and odor of each of the samples over time were compared. The sample stored at 40° C. was evaluated in accordance with the following evaluation criteria using the sample stored at 5° C. as a reference.

Evaluation Criteria:

Judgment: Evaluation

A: no change (yellowing or strange odor) from the reference

B: slight change (yellowing or strange odor) from the reference

C: change (yellowing or strange odor) from the reference

D: significant change (yellowing or strange odor) from the reference

TABLE 1

| | | Present products | | | | | | | | | Comparative products | | | |
|---|---|---|---|---|---|---|---|---|---|---|---|---|---|---|
| Component (%) | | 1 | 2 | 3 | 4 | 5 | 6 | 7 | 8 | 9 | 1 | 2 | 3 | 4 |
| (1) | beeswax | 6.0 | 6.0 | 6.0 | 6.0 | 6.0 | 6.0 | 6.0 | 6.0 | 6.0 | 6.0 | 6.0 | 6.0 | 6.0 |
| (2) | cetanol | 5.0 | 5.0 | 5.0 | 5.0 | 5.0 | 5.0 | 5.0 | 5.0 | 5.0 | 5.0 | 5.0 | 5.0 | 5.0 |
| (3) | hydrogenated oil | 5.0 | 5.0 | 5.0 | 5.0 | 5.0 | 5.0 | 5.0 | 5.0 | 5.0 | 5.0 | 5.0 | 5.0 | 5.0 |
| (4) | squalan | 30.0 | 30.0 | 30.0 | 30.0 | 30.0 | 30.0 | 30.0 | 30.0 | 30.0 | 30.0 | 30.0 | 30.0 | 30.0 |
| (5) | lipophilic glyceryl monostearate | 4.0 | 4.0 | 4.0 | 4.0 | 4.0 | 4.0 | 4.0 | 4.0 | 4.0 | 4.0 | 4.0 | 4.0 | 4.0 |
| (6) | polyoxyethylene sorbitan | 2.0 | 2.0 | 2.0 | 2.0 | 2.0 | 2.0 | 2.0 | 2.0 | 2.0 | 2.0 | 2.0 | 2.0 | 2.0 |
| | monooleate (20.E.O.) | | | | | | | | | | | | | |
| (7) | L-ascorbic acid 2-phosphate sodium salt | 3.0 | 3.0 | 3.0 | 3.0 | 3.0 | 3.0 | 3.0 | 3.0 | 3.0 | 3.0 | 3.0 | 3.0 | 3.0 |
| (8) | arginine | 0.2 | 0.2 | 0.2 | 0.4 | 0.2 | 0.6 | — | — | 0.001 | — | — | — | 0.0005 |
| (9) | urea | 0.4 | 0.4 | 0.4 | 0.8 | 0.4 | — | 0.6 | — | 0.002 | — | — | 0.002 | 0.0010 |
| (10) | triethanolamine | — | — | — | — | — | — | — | 0.6 | — | — | — | — | — |
| (11) | zinc oxide | 0.5 | 0.01 | 1.0 | 0.5 | — | — | — | — | 0.003 | — | 0.5 | — | 0.001 |
| (12) | malic acid | 0.2 | 0.2 | 0.2 | 0.2 | 0.2 | 0.2 | 0.2 | 0.2 | 0.2 | 0.2 | 0.2 | 0.2 | 0.2 |
| (13) | glycerol | 5.0 | 5.0 | 5.0 | 5.0 | 5.0 | 5.0 | 5.0 | 5.0 | 5.0 | 5.0 | 5.0 | 5.0 | 5.0 |
| (14) | 1,3-butylene glycol | 10.0 | 10.0 | 10.0 | 10.0 | 10.0 | 10.0 | 10.0 | 10.0 | 10.0 | 10.0 | 10.0 | 10.0 | 10.0 |
| (15) | antiseptic | * | * | * | * | * | * | * | * | * | * | * | * | * |
| (16) | purified water | ** | ** | ** | ** | ** | ** | ** | ** | ** | ** | ** | ** | ** |

Evaluation

| | | | | | | | | | | | | | |
|---|---|---|---|---|---|---|---|---|---|---|---|---|---|
| Storage stability examination (1) | 98.5 | 97.8 | 98.8 | 99.3 | 94.3 | 92.3 | 91.7 | 91.3 | 90.1 | 68.7 | 69.3 | 75.5 | 78.4 |
| residual rate (%) | | | | | | | | | | | | | |
| Storage stability examination (2) | A | B | A | A | B | B | B | B | B | D | D | C | C |
| outer appearance | A | A | A | A | A | B | B | B | B | D | D | D | D |
| odor | | | | | | | | | | | | | |

* appropriate amount
** balance

### Example 2

Lotion

Lotions with the composition shown in Table 2 were prepared by a common method. The lotion obtained was evaluated by the following storage stability (outer appearance) examination and storage stability (odor) examination. The results are shown in Table 2.

<Storage Stability (Outer Appearance) Examination>

Two identical sets of each sample were stored for three months in thermostatic chambers at 5° C. and 40° C., respectively. The outward appearance of both samples was evaluated in accordance with the following criteria using the sample stored at 5° C. as a reference.

<Storage Stability (Odor) Examination>

Two identical sets of each sample were stored for three months in thermostatic chambers at 5° C. and 40° C., respectively. The odor of both samples was evaluated in accordance with the following criteria using the sample stored at 5° C. as a reference.

Evaluation Criteria:

Judgment Evaluation

A: no change (yellowing or strange odor) from the reference

B: slight change (yellowing or strange odor) from the reference

C: change (yellowing or strange odor) from the reference

D: significant change (yellowing or strange odor) from the reference

TABLE 2

| | | | | | | | | Comparative | | |
|---|---|---|---|---|---|---|---|---|---|---|
| | | Present products | | | | | | products | | |
| Component (%) | | 10 | 11 | 12 | 13 | 14 | 15 | 5 | 6 | 7 |
| A | ascorbic acid sodium phosphate | 2 | 2 | 2 | 2 | 2 | 2 | 2 | 2 | 2 |
| B | arginine | 0.2 | | | 0.2 | | | | | |
| | urea | | 0.2 | | 0.2 | 1 | 0.2 | | 2 | 0.001 |
| | triethanolamine | | | 0.2 | | | | | | |
| D | alcohol | 5 | 5 | 5 | 5 | 5 | 5 | 5 | 5 | 5 |
| | 1,3-butylene glycol | 10 | 10 | 10 | 10 | 10 | 10 | 10 | 10 | 10 |
| | purified water | ** | ** | ** | ** | ** | ** | ** | ** | ** |
| | malic acid | * | * | * | * | * | * | * | * | * |
| | Storage stability (outer appearance) | B | B | B | A | B | B | D | C | D |
| | Storage stability (odor) | B | B | B | A | A | B | D | D | B |

* appropriate amount
** balance

As shown by the results in Table 2, lotions 10 to 15 of the present invention comprising a specific amount of the component (B) possessed excellent storage stability even when stored for a long period of time at a high temperature by exhibiting controlled yellowing and strange odor resulting from decomposition of the component (A) L-ascorbic acid 2-phosphate sodium salt. On the other hand, the lotion of comparative product 5 not comprising the component (B) exhibited poor storage stability, and the lotions of comparative products 6 and 7 comprising the component (B) in an amount outside the range of the present invention exhibited insufficient storage stability wherein yellowing and strange odor was present.

### Example 3

Lotion:

Lotions with the composition shown in Table 3 were prepared by a common method. Storage stability (outer appearance) examination and storage stability (odor) examination of the lotions obtained was conducted in the same manner as in Example 2. The results are shown in Table 3.

TABLE 3

| | | | | | | | | | | | Comparative | | |
|---|---|---|---|---|---|---|---|---|---|---|---|---|---|
| | | Present products | | | | | | | | | products | | |
| Component (%) | | 16 | 17 | 18 | 19 | 20 | 21 | 22 | 23 | 24 | 5 | 6 | 7 |
| A | ascorbic acid sodium phosphate | 2 | 2 | 2 | 2 | 2 | 2 | 2 | 2 | 2 | 2 | 2 | 2 |
| B | arginine | 0.2 | 0.2 | 0.2 | 0.2 | 0.2 | 0.2 | 0.2 | | | | | |
| | urea | | | | | | | | 0.2 | | | 2 | 0.001 |
| | triethanolamine | | | | | | | | | 0.2 | | | |
| C | KCl | 0.2 | | | | | | | 0.2 | 0.2 | | | |
| | NaCl | | 0.2 | | | | | | | | | | |
| | sodium sulfate | | | 0.2 | | | | | | | | | |
| | sodium carbonate | | | | 0.2 | | | | | | | | |
| | sodium hydrogen phosphate | | | | | 0.2 | | | | | | | |
| | sodium citrate | | | | | | 0.2 | | | | | | |
| | sodium lactate | | | | | | | 0.2 | | | | | |
| D | alcohol | 5 | 5 | 5 | 5 | 5 | 5 | 5 | 5 | 5 | 5 | 5 | 5 |
| | 1,3-butylene glycol | 10 | 10 | 10 | 10 | 10 | 10 | 10 | 10 | 10 | 10 | 10 | 10 |
| | purified water | ** | ** | ** | ** | ** | ** | ** | ** | ** | ** | ** | ** |
| | malic acid | * | * | * | * | * | * | * | * | * | * | * | * |
| | Storage stability (outer appearance) | A | A | A | A | A | A | A | A | A | D | C | D |
| | Storage stability (odor) | A | A | B | B | B | B | B | A | A | D | D | B |

* appropriate amount
** balance

As shown by the results in Table 3, lotions 16 to 24 of the present invention comprising specific amounts of the components (B) and (C) possessed particularly excellent storage stability even when stored for a long period of time at a high temperature and exhibited controlled yellowing and strange odor resulting from decomposition of the component (A) L-ascorbic acid 2-phosphate sodium salt.

### Example 4

Lotion:

Lotions were prepared by a common method with the composition shown in Table 4. Storage stability (outward appearance) examination and storage stability (odor) examination of the lotions obtained was conducted in the same manner as in Example 2. The results are shown in Table 4.

TABLE 4

| | | | | | | | Comparative | | |
|---|---|---|---|---|---|---|---|---|---|
| | | Present products | | | | | products | | |
| Component (%) | | 25 | 26 | 27 | 28 | 29 | 5 | 6 | 7 |
| A | ascorbic acid sodium phosphate | 2 | 2 | 2 | 2 | 2 | 2 | 2 | 2 |
| B | urea | 1 | 0.02 | 0.2 | 0.2 | 1 | | 2 | 0.001 |
| C | KCl | 0.2 | 0.2 | 2 | 0.02 | 2 | | | |
| D | alcohol | 5 | 5 | 5 | 5 | 5 | 5 | 5 | 5 |
| | 1,3-butylene glycol | 10 | 10 | 10 | 10 | 10 | 10 | 10 | 10 |
| | purified water | ** | ** | ** | ** | ** | ** | ** | ** |
| | malic acid | * | * | * | * | * | * | * | * |
| | Storage stability (outer appearance) | B | A | B | B | A | D | C | D |
| | Storage stability (odor) | A | B | B | A | B | D | D | B |

* appropriate amount
** balance

As shown by the results in Table 4, lotions 25 to 29 of the present invention comprising specific amounts of the components (B) and (C) possessed excellent storage stability even when stored for a long period of time at a high temperature and exhibited controlled yellowing and strange odor resulting from decomposition of the component (A) L-ascorbic acid 2-phosphate sodium salt.

### Example 5

Milky Lotion:

| (Component) | (%) |
|---|---|
| (1) sorbitan monostearate | 0.3 |
| (2) polyoxyethylene sorbitan monooleate (20.E.O) | 0.1 |
| (3) lipophilic glyceryl monostearate | 0.2 |
| (4) stearic acid | 0.5 |
| (5) cetanol | 0.5 |
| (6) olive oil | 3.0 |
| (7) liquid paraffin | 4.0 |
| (8) glyceryl tri-2-ethylhexanoate | 2.0 |
| (9) methylpolysiloxane | 1.0 |
| (10) hydrogenated soybean phospholipid | 0.1 |
| (11) d1-α-tocopherol acetate | 0.05 |
| (12) PEMULEN TR-1⁽*¹⁾ | 0.2 |
| (13) sodium hydroxide | 0.08 |
| (14) glycerol | 5.0 |
| (15) 1,3-butylene glycol | 7.0 |
| (16) L-ascorbic acid 2-phosphate sodium salt | 3.0 |
| (17) arginine | 0.2 |
| (18) urea | 0.4 |
| (19) malic acid | 0.2 |
| (20) zinc oxide | 0.5 |
| (21) purified water | balance |
| (22) antiseptic | appropriate amount |
| (23) perfume | appropriate amount |
| (24) ethyl alcohol | 5.0 |

⁽*¹⁾manufactured by BFGoodrich

(Method of Preparation)

A. Components (12) to (21) were mixed with heating and the mixture was allowed to stand at 70° C.

B. Components (1) to (11) were mixed with heating and the mixture was allowed to stand at 70° C.

C. B was added to A and mixed to uniformly emulsify.

D. After cooling C, components (22) to (24) were added and uniformly mixed to obtain a milky lotion.

After storing the milky lotion of Example 5 for one month in a thermostatic chamber at 50° C., a sample was analyzed by HPLC to confirm that 98.8% of the L-ascorbic acid 2-phosphate sodium salt remained. After being stored for six months in a thermostatic chamber at 40° C., the milky lotion exhibited excellent storage stability with only slight yellowing and strange odor and also possessed an excellent skin lightening effect.

### Example 6

Ointment:

| (Component) | (%) |
|---|---|
| (1) stearic acid | 18.0 |
| (2) cetanol | 4.0 |
| (3) d1-α-tocopherol | 0.2 |
| (4) vitamin A palmitate | 0.2 |
| (5) antiseptic | appropriate amount |
| (6) potassium hydroxide | 0.5 |
| (7) glycerol | 5.0 |
| (8) sodium lactate | 0.5 |
| (9) L-ascorbic acid 2-phosphate sodium salt | 20.0 |
| (10) arginine | 2.0 |
| (11) triethanolamine | 1.0 |
| (12) zinc oxide | 5.0 |
| (13) purified water | balance |

(Method of Preparation)

A. Components (6) to (13) were mixed with heating and the mixture was allowed to stand at 75° C.

B. Components (1) to (5) were mixed with heating and the mixture was allowed to stand at 75° C.

C. B was gradually added to A to obtain an ointment.

After storing the ointment of Example 6 for one month in a thermostatic chamber at 50° C., a sample was analyzed by HPLC to confirm that 93.5% of the L-ascorbic acid 2-phosphate sodium salt remained. After being stored for six months in a thermostatic chamber at 40° C., the ointment exhibited excellent storage stability with only slight yellowing and strange odor.

### Example 7

Beauty Solution:

| (Component) | (%) |
|---|---|
| (1) PEMULEN TR-2⁽*¹⁾ | 0.2 |
| (2) xanthan gum | 0.2 |
| (3) purified water | balance |
| (4) glycerol | 2.0 |
| (5) ethyl alcohol | 20.0 |
| (6) sodium hydroxide | 0.05 |
| (7) lactic acid | 0.05 |
| (8) succinic acid | 0.15 |
| (9) L-ascorbic acid 2-phosphate sodium salt | 0.1 |
| (10) arginine | 0.01 |
| (11) urea | 0.01 |
| (12) zinc oxide | 0.05 |
| (13) purified water | 10.0 |

⁽*¹⁾manufactured by BFGoodrich

(Method of Preparation)

A. Components (1) to (3) were mixed with heating and cooled.

B. Components (4) to (13) were added to A to obtain a beauty solution.

After storing the beauty solution of Example 7 for one month in a thermostatic chamber at 50° C., a sample was analyzed by HPLC to confirm that 98.1% of the L-ascorbic acid 2-phosphate sodium salt remained. After being stored for six months in a thermostatic chamber at 40° C., the beauty solution exhibited excellent storage stability and skin lightening effect with only slight yellowing and strange odor.

### Example 8

Pack:

| (Component) | (%) |
|---|---|
| (1) polyvinyl alcohol | 15.0 |
| (2) silicic anhydride | 0.5 |
| (3) polyethylene glycol | 0.5 |
| (4) polyoxypropylene methyl glucoside | 5.0 |
| (5) glycerol | 5.0 |
| (6) purified water | balance |
| (7) ethyl alcohol | 10.0 |
| (8) antiseptic | appropriate amount |
| (9) sodium hydroxide | 0.05 |
| (10) sodium citrate | 0.05 |
| (11) tartaric acid | 0.15 |
| (12) L-ascorbic acid 2-phosphate sodium salt | 1.0 |
| (13) urea | 0.1 |
| (14) triethanolamine | 0.1 |
| (15) zinc oxide | 0.5 |
| (16) purified water | 10.0 |

(Method of Preparation)

A. Components (1) to (6) were mixed and heated to 70° C. to form a solution.

B. Components (7) and (8) were mixed to form a solution.

C. B was added to A, mixed and cooled, and components (9) to (16) were uniformly dispersed in this mixture to obtain a pack.

After storing the pack of Example 8 for one month in a thermostatic chamber at 50° C., a sample was analyzed by HPLC to confirm that 92.2% of the L-ascorbic acid 2-phosphate sodium salt remained. After being stored for six months in a thermostatic chamber at 40° C., the pack exhibited excellent storage stability with only slight yellowing and strange odor.

### Example 9

Liquid Foundation

| (Component) | (%) |
|---|---|
| (1) dipentaerythritol fatty acid ester*¹ | 2.0 |
| (2) liquid paraffin | 5.0 |
| (3) stearic acid | 2.0 |
| (4) cetanol | 1.0 |
| (5) self-emulsified glyceryl monostearate | 1.0 |
| (6) 2-ethylhexyl paramethoxycinnamate | 8.0 |
| (7) antiseptic | appropriate |
| | amount |
| (8) glycerol | 5.0 |
| (9) potassium hydroxide | 0.2 |
| (10) carboxymethyl cellulose | 0.2 |
| (11) bentonite | 0.5 |
| (12) purified water | balance |
| (13) silica alumina treated titanium oxide | 6.0 |
| (14) fluorine compound treated titanium oxide fine particles | 2.0 |
| (15) color pigment | 5.0 |
| (16) mica | 2.0 |
| (17) talc | 4.0 |
| (18) zinc oxide | 1.0 |
| (19) sodium lactate | 0.3 |
| (20) malic acid | 0.2 |
| (21) L-ascorbic acid 2-phosphate sodium salt | 3.0 |
| (22) arginine | 0.2 |
| (23) urea | 0.4 |
| (24) purified water | 10.0 |

*¹Cosmol 168AR (manufactured by Nisshin OilliO Group, Ltd.)

(Method of Preparation)

A. Components (1) to (7) were heated and mixed to form a solution.

B. Components (13) to (18) were added to A, mixed uniformly, and the mixture was allowed to stand at 70° C.

C. A uniform solution of components (8) to (12) was prepared and allowed to stand at 70° C.

D. B was added to C and uniformly emulsified.

E. After cooling D, components (19) to (24) were added to obtain a liquid foundation.

After storing the liquid foundation of Example 9 for one month in a thermostatic chamber at 50° C., a sample was analyzed by HPLC to confirm that 97.3% of the L-ascorbic acid 2-phosphate sodium salt remained. After being stored for six months in a thermostatic chamber at 40° C., the liquid foundation exhibited excellent storage stability with only slight strange odor.

### Example 10

Sunscreen Milky Lotion:

| (Component) | (%) |
|---|---|
| (1) polyoxyalkylene-modified organopolysiloxane | 1.0 |
| (2) dimethylpolysiloxane | 5.0 |
| (3) octamethylcyclotetrasiloxane | 20.0 |
| (4) isotridecyl isononanoate | 5.0 |
| (5) 2-ethylhexyl paramethoxycinnamate | 5.0 |
| (6) antiseptic | appropriate amount |
| (7) perfume | appropriate amount |
| (8) silicone-treated titanium oxide fine particles | 10.0 |
| (9) silicone-treated titanium oxide | 5.0 |
| (10) polystyrene powder | 3.0 |
| (11) trimethylsiloxysilicic acid | 0.5 |
| (12) dipropylene glycol | 3.0 |
| (13) ethyl alcohol | 10.0 |
| (14) purified water | balance |
| (15) sodium chloride | 0.2 |
| (16) zinc oxide | 1.0 |
| (17) citric acid | 0.2 |
| (18) malic acid | 0.2 |
| (19) L-ascorbic acid 2-phosphate sodium salt | 3.0 |
| (20) arginine | 0.2 |
| (21) urea | 0.4 |
| (22) triethanolamine | 0.1 |
| (23) purified water | 10.0 |

(Method of Preparation)

A. A mixed dispersion of components (1) to (11) was prepared.

B. A mixed solution of components (12) to (15) was prepared.

C. B was added to A and uniformly emulsified.

D. Components (16) to (23) were added to C to obtain a sunscreen milky lotion.

After storing the sunscreen milky lotion of Example 10 for one month in a thermostatic chamber at 50° C., a sample was analyzed by HPLC to confirm that 99.0% of the L-ascorbic acid 2-phosphate sodium salt remained. After being stored for six months in a thermostatic chamber at 40° C., the sunscreen milky lotion exhibited excellent storage stability with only slight yellowing and strange odor.

### Example 11

Milky Lotion:

| (Component) | (%) |
|---|---|
| (1) sorbitan monostearate | 0.5 |
| (2) polyoxyethylene sorbitan monooleate (20.E.O.) | 0.5 |
| (3) lipophilic glyceryl monostearate | 0.5 |
| (4) stearic acid | 1.0 |
| (5) cetanol | 0.5 |
| (6) olive oil | 3.0 |
| (7) liquid paraffin | 4.0 |
| (8) glyceryl tri-2-ethylhexanoate | 2.0 |
| (9) dimethylpolysiloxane | 1.0 |
| (10) hydrogenated soybean phospholipids | 0.1 |
| (11) d1-α-tocopherol acetate | 0.05 |
| (12) acrylic acid-alkyl methacrylate copolymer | 0.2 |
| (13) sodium hydroxide | 0.08 |
| (14) glycerol | 5.0 |
| (15) 1,3-butylene glycol | 5.0 |
| (16) L-ascorbic acid 2-phosphate sodium salt | 3.0 |
| (17) arginine | 0.2 |
| (18) urea | 0.4 |
| (19) malic acid | 0.2 |
| (20) potassium chloride | 0.5 |
| (21) purified water | balance |
| (22) antiseptic | appropriate amount |
| (23) perfume | appropriate amount |
| (24) ethyl alcohol | 5.0 |

(Method of Preparation)

A. Components (12) to (21) were mixed with heating and the mixture was allowed to stand at 70° C.

B. Components (1) to (11) were mixed with heating and the mixture was allowed to stand at 70° C.

C. B was added to A and mixed to obtain uniform emulsification.

D. After cooling C, components (22) to (24) were added and uniformly mixed to obtain a milky lotion.

After storing the milky lotion of Example 11 for one month in a thermostatic chamber at 50° C., a sample was analyzed by HPLC to confirm that 94.2% of the L-ascorbic acid 2-phosphate sodium salt remained. After being stored for six months in a thermostatic chamber at 40° C., the milky lotion exhibited excellent storage stability with only slight yellowing and strange odor.

### Example 12

Beauty Solution:

| (Component) | (%) |
|---|---|
| (1) acrylic acid-alkyl methacrylate copolymer | 0.2 |
| (2) xanthan gum | 0.2 |
| (3) purified water | balance |
| (4) glycerol | 2.0 |
| (5) ethyl alcohol | 10.0 |
| (6) sodium hydroxide | 0.05 |
| (7) lactic acid | 0.05 |
| (8) succinic acid | 0.15 |
| (9) L-ascorbic acid 2-phosphate sodium salt | 0.5 |
| (10) urea | 0.1 |
| (11) sodium carbonate | 0.05 |
| (12) purified water | 10.0 |

(Method of Preparation)

A. Components (1) to (3) were mixed with heating and cooled.

B. Components (4) to (12) were added to A to obtain a beauty solution.

After storing the beauty solution of Example 12 for one month in a thermostatic chamber at 50° C., a sample was analyzed by HPLC to confirm that 97.7% of the L-ascorbic acid 2-phosphate sodium salt remained. After being stored for six months in a thermostatic chamber at 40° C., the beauty solution exhibited excellent storage stability with only slight yellowing and strange odor.

### Example 13

Pack:

| (Component) | (%) |
|---|---|
| (1) polyvinyl alcohol | 15.0 |
| (2) silicic anhydride | 0.5 |
| (3) polyethylene glycol | 0.5 |
| (4) glycerol | 5.0 |
| (5) purified water | balance |
| (6) ethyl alcohol | 10.0 |
| (7) antiseptic | appropriate amount |
| (8) sodium hydroxide | 0.05 |
| (9) sodium citrate | 0.2 |
| (10) tartaric acid | 0.15 |
| (11) L-ascorbic acid 2-phosphate sodium salt | 1.0 |
| (12) urea | 0.1 |
| (13) triethanolamine | 0.1 |
| (14) purified water | 10.0 |

(Method of Preparation)

A. Components (1) to (5) were mixed and heated to 70° C. to form a solution.

B. Components (6) and (7) were mixed to form a solution.

C. B was added to A, mixed and cooled, and components (8) to (14) were uniformly dispersed in this mixture to obtain a pack.

After storing the pack of Example 13 for one month in a thermostatic chamber at 50° C., a sample was analyzed by HPLC to confirm that 96.8% of the L-ascorbic acid 2-phosphate sodium salt remained. After being stored for six months in a thermostatic chamber at 40° C., the pack exhibited excellent storage stability with only slight yellowing and strange odor.

### Example 14

Liquid Foundation:

| (Component) | (%) |
|---|---|
| (1) dipentaerythritol fatty acid ester*¹ | 2.0 |
| (2) liquid paraffin | 5.0 |
| (3) stearic acid | 2.0 |
| (4) cetanol | 1.0 |
| (5) self-emulsified glyceryl monostearate | 1.0 |
| (6) 2-ethylhexyl paramethoxycinnamate | 8.0 |
| (7) antiseptic | appropriate |
| | amount |
| (8) glycerol | 5.0 |
| (9) potassium hydroxide | 0.2 |
| (10) carboxymethyl cellulose | 0.2 |
| (11) bentonite | 0.5 |
| (12) purified water | balance |
| (13) silica alumina treated titanium oxide | 6.0 |
| (14) fluorine compound treated titanium oxide fine particles | 2.0 |
| (15) color pigment | 5.0 |
| (16) mica | 2.0 |
| (17) talc | 4.0 |
| (18) malic acid | 0.2 |
| (19) L-ascorbic acid 2-phosphate sodium salt | 2.0 |
| (20) arginine | 0.5 |
| (21) urea | 0.3 |
| (22) purified water | 10.0 |

*¹Cosmol 168AR (manufactured by Nisshin OilliO Group, Ltd.)

(Method of Preparation)

A. Components (1) to (7) were heated and mixed to form a solution.

B. Components (13) to (17) were added to A, mixed uniformly, and the mixture was allowed to stand at 70° C.

C. A uniform solution of components (8) to (12) was prepared and allowed to stand at 70° C.

D. B was added to C and uniformly emulsified.

E. After cooling D, components (18) to (22) were added to obtain a liquid foundation.

After storing the liquid foundation of Example 14 for one month in a thermostatic chamber at 50° C., a sample was analyzed by HPLC to confirm that 92.1% of the L-ascorbic acid 2-phosphate sodium salt remained. After being stored for six months in a thermostatic chamber at 40° C., the liquid foundation exhibited excellent storage stability with only slight yellowing and strange odor.

### Example 15

Sunscreen Milky Lotion:

| (Component) | (%) |
|---|---|
| (1) polyoxyalkylene-modified organopolysiloxane | 1.0 |
| (2) dimethylpolysiloxane | 5.0 |
| (3) octamethylcyclotetrasiloxane | 20.0 |
| (4) isotridecyl isononanoate | 5.0 |
| (5) 2-ethylhexyl paramethoxycinnamate | 5.0 |
| (6) antiseptic | appropriate amount |
| (7) perfume | appropriate amount |
| (8) silicone treated titanium oxide fine particles | 10.0 |
| (9) silicone-treated titanium oxide | 5.0 |
| (10) polystyrene powder | 3.0 |
| (11) trimethylsiloxysilicic acid | 0.5 |
| (12) dipropylene glycol | 3.0 |
| (13) ethyl alcohol | 10.0 |
| (14) purified water | balance |
| (15) sodium chloride | 1.0 |
| (16) malic acid | 0.5 |
| (17) L-ascorbic acid 2-phosphate sodium salt | 5.0 |
| (18) arginine | 0.2 |
| (19) urea | 0.4 |
| (20) diethanolamine | 0.1 |
| (21) purified water | 10.0 |

(Method of Preparation)

A. A mixed dispersion of components (1) to (11) was prepared.

B. A mixed solution of components (12) to (15) was prepared.

C. B was added to A and uniformly emulsified.

D. Components (16) to (21) were added to C to obtain a sunscreen milky lotion.

After storing the sunscreen milky lotion of Example 15 for one month in a thermostatic chamber at 50° C., a sample was analyzed by HPLC to confirm that 93.5% of the L-ascorbic acid 2-phosphate sodium salt remained. After being stored for six months in a thermostatic chamber at 40° C., the sunscreen milky lotion exhibited excellent storage stability with only slight yellowing and strange odor.

### INDUSTRIAL APPLICABILITY

The cosmetic of the present invention comprising L-ascorbic acid 2-phosphate sodium salt prevents decomposition of the L-ascorbic acid 2-phosphate sodium salt even when stored for a long period of time at a high temperature. Therefore, the cosmetic of the present invention can be suitably used as cosmetic comprising L-ascorbic acid 2-phosphate sodium salt as an active component, for example, a skin lightening cosmetic and the like.

1. A cosmetic comprising the following components (A) and (B):
(A) L-ascorbic acid 2-phosphate sodium salt,
(B) one or more compounds selected from the group comprising arginine, urea, and triethanolamine,
wherein the mass ratio of (A):(B) is 1:0.001 to 0.5.

2. The cosmetic according to claim 1 further comprising a component (C) zinc oxide or a salt which produces an alkali metal ion in water, wherein the mass ratio of (A):(C) is 1:0.001 to 1.

3. The cosmetic according to claim 1 or claim 2 comprising 0.001 to 20 mass % of the component (A).

4. The cosmetic according to any one of claims 1 to 3, wherein the component (B) comprises arginine and urea.

5. The cosmetic according to any one of claims 2 to 4, wherein the salt which produces an alkali metal ion in water of the component (C) is potassium chloride or sodium chloride.

6. The cosmetic according to any one of claims 1 to 5 used as an aqueous cosmetic or emulsion cosmetic.

7. The cosmetic according to any one of claims 1 to 6 having a pH of 7 to 9.

8. The cosmetic according to any one of claims 1 to 7 used as a skin lightening cosmetic or anti-aging cosmetic.
